(19) United States
(12) Patent Application Publication
(10) Pub. No.: US 2026/0204366 A1
(43) Pub. Date: Jul. 16, 2026

(54) USER INTERFACE FOR MATERIALS AND MATERIAL PROPERTIES PREDICTION USING A MACHINE LEARNING MODEL

(71) Applicant: Dow Global Technologies LLC, Midland, MI (US)

(72) Inventors: Fabio Aguirre Vargas, Lake Jackson, TX (US); Sukrit Mukhopadhyay, Midland, MI (US); Bo Shuang, Missouri City, TX (US); Marcie E. Kaiser, St. Charles, MI (US)

(21) Appl. No.: 19/137,003

(22) PCT Filed: Dec. 20, 2023
(86) PCT No.: PCT/US2023/085094

§ 371 Date: Jun. 9, 2025

Related U.S. Application Data
(60) Provisional application No. 63/433,986, filed on Dec. 20, 2022.

(51) Int. Cl. G16C 60/00; G16C 20/30; G16C 20/70; G16C 20/80

(52) U.S. Cl. CPC G16C 60/00; G16C 20/30; G16C 20/70; G16C 20/80

(57) ABSTRACT

Machine learning can be used to predict formulations for an output formulation. The machine learning can be implemented by a machine learning model, which employs a forward model and an inverse model. A user interface can be used to gather raw materials selections and output formulation property selections. The selections can be used to generate formulations that comply with selections using the ML model.

### TECHNICAL FIELD

Embodiments relate to predicting and visualizing materials and output formulation properties using a computational model. Such techniques can be particularly useful to predict output formulation properties and raw materials in view of output formulation property selections and raw materials selections provided by a user.

### BACKGROUND

Artificial Intelligence (AI) refers to the ability to improve a machine through “learning” such as by storing patterns and/or examples, which can be utilized to take actions at a later time. Examples of AI include artificial neural networks (ANNs), decision trees, support vector machines, and partial least squares; these examples can be broadly classified as machine learning (ML) algorithms. ANNs are networks that can process information by modeling a network of neurons, such as neurons in a human brain, to process information (e.g., stimuli) that has been sensed in a particular environment. Similar to a human brain, neural networks typically include a multiple neuron topology (e.g., that can be referred to as artificial neurons). An ANN operation refers to an operation that processes inputs using artificial neurons to perform a given task. Example tasks that can be processed by performing ML include machine vision, speech recognition, machine translation, social network filtering, and/or medical diagnosis.

### SUMMARY OF THE DISCLOSURE

An aspect is directed to predicting materials and output formulation properties using a ML model. The ML model can include a forward model (a ML model that predicts the properties of a formulated material from the composition, including raw materials and their weight fractions) and an inverse model (a ML model that predicts the raw materials and their weight fractions from the properties of formulated materials).

An interface can be used to receive selections from a user. The selections can include an output formulations selection, an output formulation properties selection, and raw materials and weight fractional selection. The output formulations selection, the output formulation properties selection, and the raw materials selection can be provided to the ML model. The ML model can predict raw materials composition and output formulation properties, which can be provided to a user through a user interface. The formulation can also include weight percentage values for each of the raw materials. The formulation can comply with the selected design space of raw materials and the selected output formulation properties.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 illustrates an example computing system for predicting raw materials, their weight fractions, and output formulation properties using a ML model in accordance with some embodiments of the present disclosure.

FIG. 2A illustrates an interface for choosing different market applications in accordance with some embodiments of the present disclosure.

FIG. 2B illustrates an interface for choosing units and data models (which includes inverse ML models) in accordance with some embodiments of the present disclosure.

FIG. 2C illustrates an interface for choosing output formulation properties in accordance with some embodiments of the present disclosure.

FIG. 3A illustrates an interface for constraining the design space for compositions in accordance with some embodiments of the present disclosure.

FIG. 3B illustrates an interface constraining the design space for compositions to produce an output formulation composition and properties in accordance with some embodiments of the present disclosure.

FIG. 4 illustrates an interface for choosing the composition of formulations (materials and their weight fractions) in accordance with some embodiments of the present disclosure.

FIG. 5 illustrates an interface for predicting properties using a ML model and visualizing those with the historical formulations and tabulating them to extract in an excel format in accordance with some embodiments of the present disclosure.

FIG. 6 is a flow diagram for operating a user interface to predict raw materials, their compositions, and output formulation properties using a ML model in accordance with some embodiments of the present disclosure.

FIG. 7 is a block diagram of an example computer system in which embodiments of the present disclosure may operate.

### DETAILED DESCRIPTION

Machine learning (ML) can apply ANNs, decision trees, support vector machines, and partial least squares, among other ML models, to accomplish tasks once thought impossible for a computer to perform. Machine learning including deep learning (e.g., ANNs) can be used to make predictions of output formulation properties and raw materials of an output formulation. As used herein, an output formulation can include a material that is made through a reactive mixture of raw materials (e.g., input materials). The formulation can include combinations of raw materials, which can include chemical reactions, physical interactions, or no reactions. The output formulation can have a number of properties, which comprise output formulation properties. The output formulation can be a polyurethane foam, for example. The raw materials can include monomers, polymers, chemicals, fillers, base materials, masterbatch materials, compounds, etc. The output formulation properties can include, for example, a gel time, a density free rise, and/or a composition strength, among other properties of the output formulation.

However, generating predictions for output formulation properties can involve a detailed knowledge of forward models or inverse models that utilize ML. Generating predictions can also be slow given that multiple models can be run sequentially prior to generating the predictions. Furthermore, such models are complex and may not be able to be run by chemical formulators who may not have the requisite programming skills or the time required to use the models to get the predictions they desire. In some environments, permissions may be required to run the models due to the computing time and energy needed. In some instances, the forward and inverse models may need to be run in a particular sequence to get the right set of experiments.

Aspects of the present disclosure address the above and other deficiencies by utilizing a ML model, which comprises both a forward model and an inverse model. Implementing a forward model and/or an inverse model, which comprise the ML models, can allow for predictions to be made based on output formulation property selections and/or raw materials selections. The ML model can also be implemented in a cloud environment, which can allow for the forward model and the inverse model to be executed concurrently allowing for a shortened time to deliver the needs of a scientist as compared to implementing the forward model and the inverse model sequentially.

As used herein, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected and, unless stated otherwise, can include a wireless connection.

As will be appreciated, elements shown in the various embodiments herein can be added, exchanged, and/or eliminated so as to provide a number of additional embodiments of the present disclosure. In addition, as will be appreciated, the proportion and the relative scale of the elements provided in the figures are intended to illustrate certain embodiments of the present invention and should not be taken in a limiting sense.

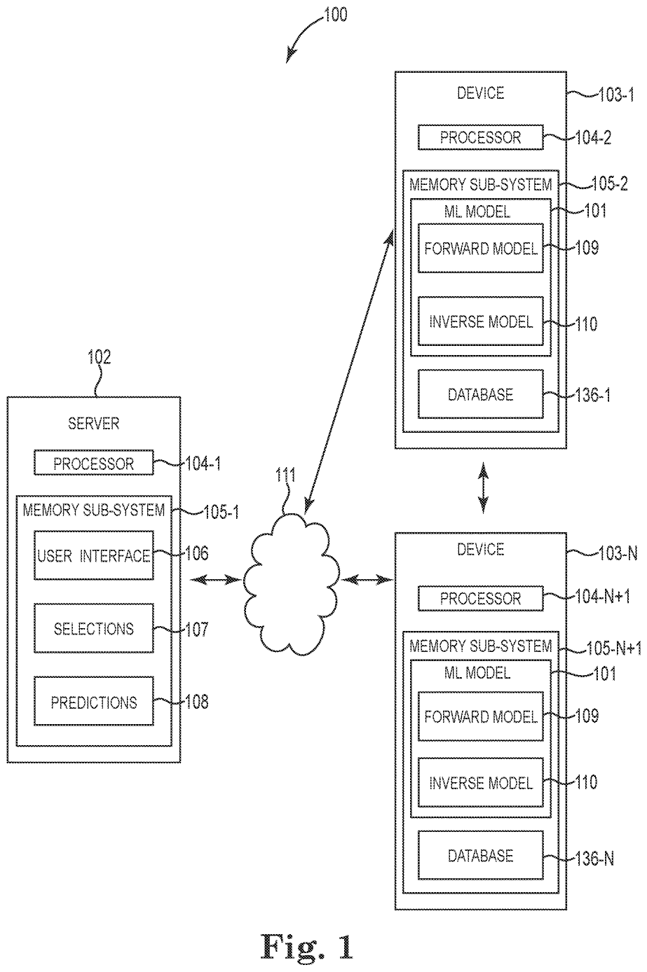

FIG. 1 illustrates an example computing system 100 for predicting raw materials and output formulation properties using a ML model in accordance with some embodiments of the present disclosure. The computing system 100 can comprise a server 102 and cloud devices 103-1, 103-N (e.g., devices 103-1, 103-N), referred to herein as cloud devices 103.

The computing system 100, the server 102, and the cloud devices 103 can comprise hardware, firmware, and/or software configured to generate predictions of raw materials and their weight fractions and output formulation properties for an output formulation (e.g., formulated material such as polyurethane foam). As used herein, a weight fraction describes a fractional representation of a weight of a raw material in a formulation. For example, a weight faction of 5 represent that half of a formulation is composed of a particular raw material. Although the examples provided herein are given in terms of weight fractions, other measurements can be used such a volume fraction and/or weight of a raw material. The volume fraction can represent a volume representation of a raw material in a formulation. The server 102 and the cloud devices 103 can further include memory sub-systems 105-1, 105-2, 105-N+1 (e.g., a non-transitory machine readable medium (MRM)), referred to herein as memory sub-systems 105, on which may be stored instructions for executing a user interface 106, instructions for executing a ML model 101, user selections 107, and/or predictions 108 generated by the ML model. Although the following description refers to a processing device and a memory device, the description may also apply to a system with multiple processing devices and multiple memory devices. In such examples, the instructions may be distributed (e.g., stored) across multiple memory devices and the instructions may be distributed (e.g., executed by) across multiple processing devices.

The memory sub-systems 105 may comprise memory devices. The memory devices may be electronic, magnetic, optical, or other physical storage device that store executable instructions. One or both of the memory devices may be, for example, non-volatile or volatile memory. In some examples, one or both of the memory device is a non-transitory MRM comprising random access memory (RAM), an Electrically-Erasable Programmable read only memory (ROM) (EEPROM), a storage drive, an optical disc, and the like. The memory sub-systems 105 may be disposed within a controller, the server 102, and/or the cloud devices 103. In this example, the user interface 106 can be “installed” on the server 102. The memory sub-systems 105 can be portable, external or remote storage mediums, for example, that allow the server 102 and/or the cloud devices 103 to receive user selections. In this situation, the user interface 106 may be part of an “installation package.” As described herein, the memory sub-systems 105 can be encoded with executable instructions for providing a user interface 106 via hardware comprising a monitor and/or a projector, among other means of providing a user interface.

The server 102 can execute the user interface 106 using the processor 104-1 also referred to herein as processing device 104-1 and/or a processing resource. Executing the user interface 106 can include executing instructions for generating the user interface. Executing the user interface 106 can also include displaying the user interface 106. The user interface 106 (e.g., user interface instructions) can be stored in the memory sub-system 105-1 prior to being executed by the processing device 104-1. The user interface 106 can be executed to receive selections 107 (e.g., user selections) and to generate predictions 108.

Examples of the selections 107 include output formulation selections, raw material selections, and/or output formulation properties selections. The selections 107 can be provided via the user interface 106. Processor 104-1 can receive the selections 107 and can provide the selections 107 to the cloud devices 103.

As used herein, cloud computing and/or a cloud environment is an on-demand availability of computer system resources utilizing a network 111. The resources made available for cloud computing can include cloud storage and cloud computing power. Cloud computing can rely on resource sharing. Resource sharing includes the use of the resources of the cloud devices 103 to predict raw materials and to predict output formulation properties. The cloud devices 103 can be implemented in different physical locations and/or a same location and across different devices and/or a same device. The cloud devices 103 can utilize different physical resources such as separate processors (e.g., processors 104-2, 104-N+1) and separate memory sub-systems (e.g., memory sub-systems 105-2, 105-N+1), which can be shared by the ML model to generate the predictions 108.

The cloud devices 103 can store instructions for executing a ML model (e.g., ML model instructions 101). The instructions for executing a ML model 101 can also include instructions for executing a forward model 109 and an inverse model 110 as the ML model 101 is comprised of the forward model 109 and the inverse model 110. The ML model 101 can utilize the forward model 109 and/or the inverse model 110 to generate predictions for the raw materials and/or predictions for the output formulation properties.

The forward model 109 can generate output formulation property predictions from raw materials (e.g., selected raw materials) and their weight fractions provided by a user via the interface 106. The inverse model 109 can generate raw materials and their weight fraction predictions from targeted formulation properties provided by a user via the interface 106. The forward model 109 and the inverse model 110 can be implemented in different devices. For example, the forward model 109 can be implemented in the cloud device 103-1 while the inverse model 110 is implemented in the cloud device 103-N.

The processor 104-1 can provide raw material selections, their weight fractions, and output formulation property selection to the ML model 101. The processor 104-2 can execute the forward model 109 to generate output formulation property predictions from the raw material selections provided by the processor 104-1. A byproduct of generating the output formulation property predictions can be the identification (e.g., prediction) of raw materials and their weight fractions including the raw materials selections and their weight fractions that can be used to generate the predicted output formulation properties. The processor 104-2 can execute the forward model 109 concurrently with the execution of the inverse model 110 by the processor 104-N+1. The output formulation property predictions and the raw materials predictions and their weight fractions identified can be referred to as a first formulation. In various instances, the forward model 109 can generate multiple first formulations each being distinct from the others.

The processor 104-N+1 can execute the inverse model 110 to generate at least one set of raw materials and their weighted fractions prediction using the output formulation property selections. A byproduct of generating predictions for the raw materials and their weight fractions can include identifying output formulated properties including the output formulation properties. The inverse model 110 can generate multiple sets of formulations (e.g., containing raw materials and their weight fractions) each being distinct from the others.

The ML model 101 can compare the first formulations to the second formulations to identify formulations having the raw materials selections and the output formulation property selections. The identified formulations can be provided to the server 102. The processor 104-1 can receive the identified formulations and can display the identified formulations via the user interface 106. In various instances, the ML model 101 can rank the identified formulations based on the raw material selections, their weight fraction selections, and the output formulation property selections prior to providing the identified formulations to the server 102.

In various instances, the ML model 101 can cause the first formulations and the second formulations to be provided to a same cloud device to allow for a selection of formulations. For example, the ML model 101 can cause the first formulations to be provided to the cloud device 103-N or the ML model 101 can cause the second formulations to be provided to the cloud device 103-1. In various instances, the ML model 101 can cause the first formulations and the second formulations to be provided to a different cloud device (not shown). Providing the first formulations and the second formulations to a different cloud device allows the cloud devices 103-1, 103-N to continue to execute the forward model 109 and the inverse model 110. Data can be provided between the server 102 and the cloud devices 103 utilizing a network 111.

In various instances, the forward model 109 and the inverse model 110 can utilize machine learning operations to generate the predictions. The forward model 109 and the inverse model 110 can also be utilized to generate formulations based on the selections 107. For example, the forward model 109 and the inverse model 110 can search historical formulations to identify appropriate formulations. The server 102 can provide the selection 107 (e.g., raw materials and their weight fraction selections and output formulation property selections) to the forward model 109 and the invers model 110 that identifies whether the forward model 109 and the inverse model 110 are to search historical formulations and/or are to predict formulations. In various instances, the server 102 can provide indication that one of the forward model 109 and the inverse model 110 is to search historical formulations while the other is to predict new formulations. The historical formulations can be stored in database (e.g., databases 136-1, 136-N) for example. The databases 136-1, 136-N can comprise hardware, firmware, and/or software, which can be used to store and retrieve historical formulations. In various instances, it may be faster to retrieve historical formulations from the databases 136-1, 136-N than it may be to generate predictions utilizing ML. The forward model 109 and the inverse model 110 can be configured to search the historical formulation and can be configured to generate formulation predictions.

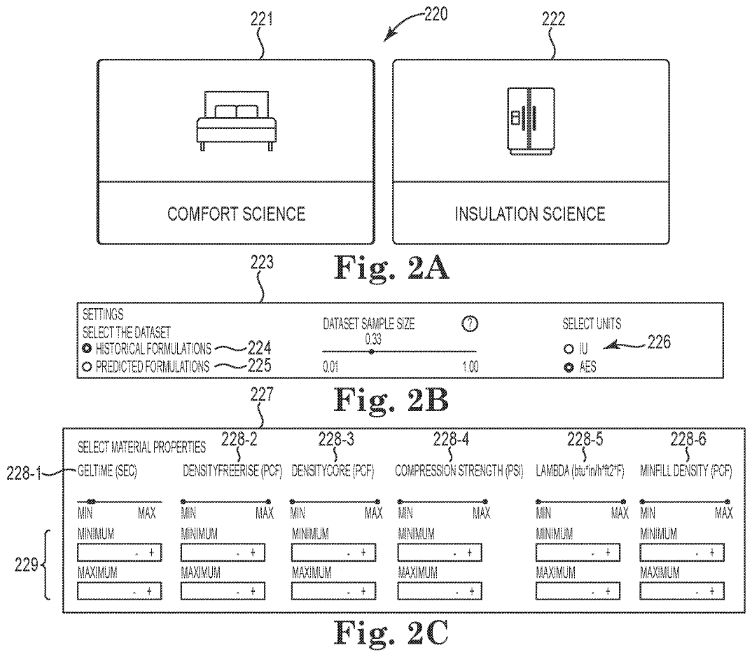

FIG. 2A illustrates an interface 220 for choosing different market applications in accordance with some embodiments of the present disclosure. The interface 220 can be configured to receive a selection of a market segment. For example, an option can be presented to a user to select between a comfort science 221 market segment and an insulation science 222 market segment.

The comfort science 221 market segment can include output formulations (e.g., formulated materials) used for comfort. The insulation science 222 market segment can include output formulations used for insulation. In some instances, a particular class of output formulation (e.g., polyurethane foam) may serve more than one market segment, but the specific types of output formulation may have different properties and/or requirements for the different market segments. For example, output formulations used for comfort can have different properties from the output formulation used for insulation. Identifying output formulations used for comfort or insulation can help the ML model reduce the search space used to generate predictions. The selection between the comfort science 221 market segment and the insulation science 222 market segment can be referred to as an output formulation selection. The output formulation selection can describe whether the output formulation (e.g., polyurethane foam product) is used for comfort or insulation.

FIG. 2B illustrates an interface for choosing units and data models (which includes inverse ML models) in accordance with some embodiments of the present disclosure. A dataset selection 223 can be a historic formulation 224 or a predicted formulation 225.

The historic formulations 224 can comprise a dataset comprised of formulations that have been experimentally verified. The historic formulations 224 can be smaller than the predicted formulations 225.

The predicted formulations 225 can be predicted using ML models and can consist of raw materials, their weight fractions, and output formulation properties. However, the selection of the predicted formulations 225 can also include a search of previously predicted formulations that have been saved in the database (e.g., databases 136-1, 136-N of FIG. 1). The predicted formulations 225 can be generated utilizing machine learning operations, for example. The forward model and the inverse model of the ML model can be configured to search predicted formulations 225 and/or generate predicted formulations 225. In the event that predicted formulations 225 is selected, the user can be prompted to select a dataset sample size, which can give the user some control over the quantity of predicted results that will be generated, viewed, and/or some control over any processing time that may be used for such predictions.

Units 226 can also be selected. For example, a selection can be made between the International Units (IU) and American Engineering System (AES) units. The units selected can be used to display values corresponding to the selected raw materials and/or the selected output formulation properties. The units selected can also be used to display values for predicted raw materials and/or predicted output formulation properties.

FIG. 2C illustrates an interface for choosing output formulation properties in accordance with some embodiments of the present disclosure. FIG. 2C shows output formulation properties 227 (e.g., a pane 227 comprising output formulation properties 227). The output formulation properties 227 can be selected within a certain range. The certain range can be predefined. Examples of the output formulation properties 227 can include a gel time property 228-1, a density free rise property 228-2, a density core property 228-3, a compression strength property 228-4, a lambda property 228-5, and/or a min fill property (e.g., min fill density property) 228-6, among other possible properties.

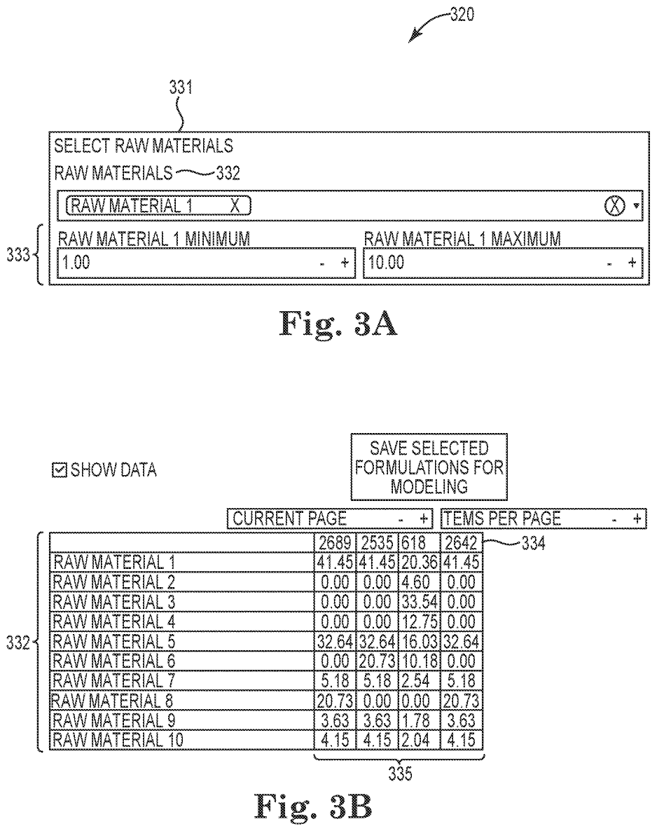

Each of the output formulation properties 227 can be associated with minimum and maximum values 229. The user can select a minimum and/or a maximum value for each of the output formulation properties 227. The minimum and/or maximum values can be utilized by the ML model to generate predictions and/or search historical formulations. The interface comprising the output formulation properties 227 can provide appropriate interface elements for selecting a property, a minimum value, and/or a maximum value. The selected output formulation properties 227 can be used by an inverse model. FIG. 3A illustrates an interface for constraining the design space for compositions in accordance with some embodiments of the present disclosure.

FIG. 3A illustrates an interface 320 for constraining the design space for compositions in accordance with some embodiments of the present disclosure. The interface 320 includes a material selection pane 331, which allows for a selection of materials 332 (e.g., raw materials).

A number of possible materials 332 can be provided via the interface 320 using a drop-down menu, for example. A weight fraction of raw materials 333 (e.g., minimum and/or maximum values) can also be selected. The material selection can be used for a forward model.

The selected raw materials 332 and the selected output formulation properties can be provided to the ML model. The selected raw materials 332 and the selected output formulation properties can also be visualized in a table format and can be extracted in an excel format. The selected raw materials 332 and the selected output formulation properties can be used by the forward model and the inverse model to generate a first number of formulations and a second number of formulations. The selected raw materials 332 and the selected output formulation properties can also be used by the ML model to select formulations from the first number of formulations and the second number of formulations for presenting via the interface 320. FIG. 3B illustrates an interface constraining the design space for compositions to produce an output formulation composition and properties in accordance with some embodiments of the present disclosure. Interface 320 displays the selected formulations 334.

The selected formulations 334 include the materials 332 and the weight fraction 335 of the materials 332. The weight fraction 335 of the material can describe a quantity of the materials 332 in the selected formulations 334. For example, the weight fraction 335 can be expressed as a percentage by weight of the materials 332 in the selected formulations 334. The selected formulations 334 can be displayed responsive to a selection of the raw materials 332 and/or the selection of the output formulation properties.

In various instances, the materials 332 of the selected formulation 334 can comprise more materials than the raw materials selections. The materials 332 can include at least the raw materials selections. For example, the user may not know all of the raw materials that will be used in a formulation for a given output formulation, but the user may have one or a few materials that are desired to be used in the formulation due to availability, user specifications, or other reasons. The selected formulations 334 can be saved. The interface can provide for a modification of the raw materials 332, which can cause change to the selected formulations 334 and their properties.

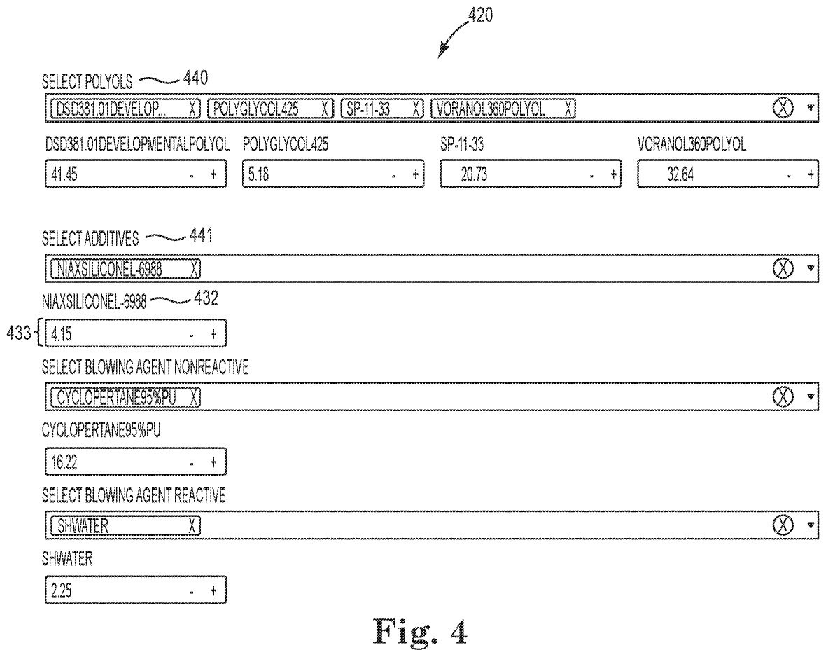

FIG. 4 illustrates an interface 420 for choosing the composition of formulations (materials and their weight fractions) in accordance with some embodiments of the present disclosure. The interface 420 provides the capability for modification of the raw materials and their weight fractions provided in the selected formulation. For example, raw material A can be changed to raw material B. As another example, an additional raw material can be added to the formulation or a particular raw material can be deleted from the formulation. The interface 420 provides for the modification of the weight fractions of the raw material selections. For example, an amount of a particular raw material (e.g., weight percentage) can be changed to a different amount in a formulation.

The interface 420 also provides for inclusion 441 of additives that were not included in the selected formulations. For example, a Silicone surfactant L-6988 material 432 can be selected for inclusion in the formulation. An amount 433 can also be selected for the additive 432. The updated raw material selections and their corresponding weight fraction can be provided to the ML model. The ML model can generate updated formulations, which can be provided back to the server. The server can display the updated formulation via the interface 420.

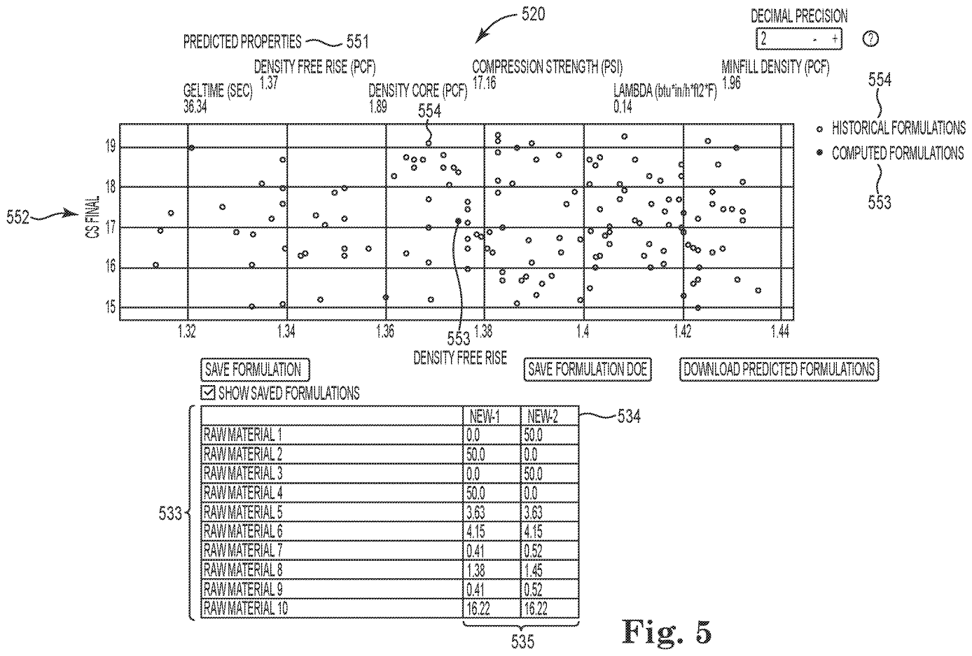

FIG. 5 illustrates an interface 520 for predicting properties using a ML model and visualizing those with the historical formulations and tabulating them to extract in an excel format in accordance with some embodiments of the present disclosure. The interface 520 can display the output formulation properties 551 corresponding to one or more formulations selected in view of the raw materials selections and the weight fractions. The selected formulations can also be visualized using graph 552. The graph 552 can provide a visual representation of the value of the output formulation property corresponding to the raw materials of the selected formulations. The graph 552 can show the value of the output formulation property from the selected formulation(s) 553 alongside corresponding values of the output formulation property for output formulations. The graph can be generated by a computing device (e.g., server) as opposed to the cloud devices. The interface 520 can also display the formulations 534 comprising the raw materials 533 and the corresponding amounts 535. The formulations 534 can be updated based on the updated selections. The updated selections (e.g., raw materials and output formulation properties) can be provided to the ML model and the ML model can provide the updated formulations 534. The server can generate the graph 552 from the formulations 534.

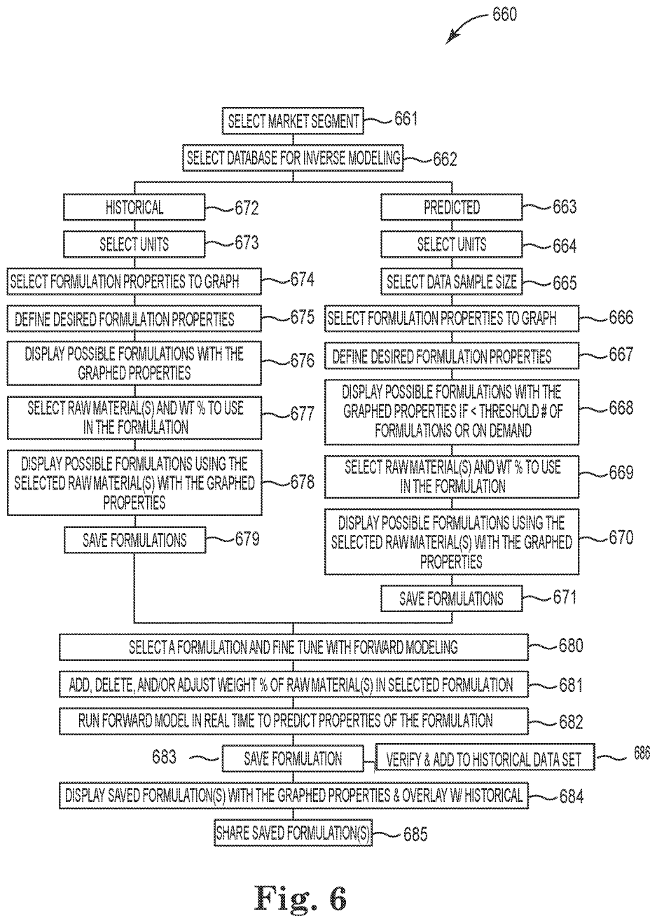

FIG. 6 is a flow diagram 660 for operating a user interface to predict raw materials, their compositions, and output formulation properties using a ML model in accordance with some embodiments of the present disclosure. The user interface can help the user find a good starting point for the ML modeling and run the ML modeling without being a data science expert. The flow diagram 660 can begin at 661. At 661, a market segment can be selected. An example user interface for selection of a market segment is illustrated in FIG. 2A at 221, 222. At 662, a database can be selected from inverse modeling. The selected database can also be utilized for forward modeling. The selected database can be used, via the user interface, to search for a formulation that produces an output formulation, which is close to the desired output formulation. Then, the user can make edits to the formulation to create an updated formulation that produces an output formulation, whose properties can be predicted and compared with the desired ranges. An example user interface for selection of a database is illustrated in FIG. 2B at 224, 225.

In view of a predicted 663 selection for the database, the flow diagram 660 can continue to 664. At 664, units can be selected. An example user interface for selection of units is illustrated in FIG. 2B at 226. In some embodiments, a graph may not be created automatically (e.g., if the selected database is so large that delays in rendering the graph would reduce the user experience). At 665, a data sample size can be selected (e.g., to reduce the amount of data that will be considered, thereby reducing any lag in rendering the graph(s)). An example user interface for data sample size selection is illustrated in FIG. 2B. At 666, output formulation properties to include in one or more graphs can be selected. At 667, desired output formulation properties can be defined. For example, maximum and/or minimum values (e.g., a range of values) can be selected for the output formulation properties. An example user interface for output formulation property selection is illustrated in FIG. 2C.

At 668, the possible formulations (e.g., selected formulations) can be displayed as a graph. The graph can be displayed utilizing graph properties including the output formulation properties that were selected at 666. The possible formulations can be displayed if they are fewer in number than a threshold number of formulations (e.g., based on a predefined threshold, or based on the data sample size selected at 665). The possible formulations can be graphed on demand based on a user request regardless of the threshold. By way of example, the user request can be received via a click of a button on the user interface.

At 669, raw materials and weight fractions can be selected and amounts of the raw materials can be defined to use in the formulation. An example user interface for selecting raw materials is illustrated in FIG. 3A. The amounts can include a maximum and/or a minimum value. In various instances, the maximum value and the minimum value can be a same value. The user may wish to add a raw material to the formulation or specify some particular weight fraction for a raw material, for example, based on a raw material that the user has or commonly has on hand. Any additional input from the user in this regard is likely to narrow the field of possible formulations.

At 670, the possible formulations can be displayed using the selected raw material and the graphed properties. At 671, the formulation can be saved allowing for the formulations to be retrieved at a later time. The user can download the formulation information.

Returning to the database selection at 662, at 672, a historical database can be selected. At 673, units can be selected analogously to 665. At 674, output formulation properties can be selected to graph analogously to 666. At 675, output formulation properties can be defined analogously to 667.

At 676, possible formulations can be displayed with the graphed properties. In contrast to the display option at 668, the display option at 676 for the historical database does not have a threshold criterion. If the historical database becomes greater than a threshold, then a threshold criterion can be implemented. At 677, raw materials and corresponding weight fractions can be selected for use in the formulations analogously to 669. At 678, the possible formulations can be displayed using the selected raw materials with the graphed properties analogously to 670. At 679, the formulations can be saved analogously to 671.

Responsive to saving the formulations at 671 and/or 679, a formulation can be selected and fine-tuned with forward modeling at 680. Selection of the formulation can cause the formulation raw materials to be displayed along with their weight fractions. An example user interface for fine tuning the formulation is illustrated in FIG. 3B. At 681, raw materials can be added or deleted for the selected formulation. The weights percentage of the raw materials of the formulations can also be adjusted for the selected formulation. An example user interface for the operations at 681 is illustrated in FIG. 4. If the user makes any changes with respect to the raw materials, the ML model (e.g., the forward model portion of the ML model) can run in real time to predict the properties of the formulation, which result from the changes to raw materials of the formulation and their weight fractions as indicated at 682. At 683, the formulations (e.g., updated formulations) can be saved. At 684, the saved formulations can be displayed with graphed properties and overlayed with historical formulations. An example of such a graph is illustrated in FIG. 5 at 552. At 685, the saved formulation can be shared with one or more users. At 686, the predicted output formulated properties can be experimentally verified and added to the historical data set. The output formulated properties and the raw materials and their weight fractions can be optionally included in the predicted database (e.g., dataset). The predicted output formulated properties can be experimentally verified and added to the historical data set prior to saving the formulation at 683 or after saving the formulation at 686.

In various examples, an output formulation selection can be received at a processing resource. For example, the output formulation selection can include one of a comfort science and/or insulation science.

A number of output formulation property selections can be received at the processing resource. A number of raw materials selections can be received at the processing resource. The output formulation selection, the number of output formulation property selections, and the number of raw material selection can be received via a user interface,

The output formulation selection, the number of output formulation property selections, and the number of raw material selections can be provided utilizing a network interface coupled to the processing resource to a machine learning model. The ML model can be described as a ML model when the ML model employs machine learning operations to generate predictions. The ML model can comprise a forward model and an inverse model that predict output formulation properties and raw materials. In various examples, the ML model can be implemented in a cloud environment. For example, the ML model can be implemented in one or more cloud devices.

The predicted output formulation properties and the predicted raw materials can be received for an output formulation from the ML model. The predicted output formulation properties and the predicted raw materials can be provided to the user interface for display.

In various examples, feedback material properties can be received responsive to generating the output formulation using the predicted raw materials. For example, the output formulation can be generated using the predicted raw materials. The properties of the output formulation can be measured and provided to the ML model as feedback. The ML model can use the feedback for training. For example, the forward model and the inverse model can be trained using the feedback.

Responsive to receipt of the raw materials, the updated raw materials and corresponding weights percentages of the updated raw materials can be received via the user interface. The updated raw materials can be provided to the ML model utilizing the network interface. Updated predicted output formulation properties and updated predicted raw materials can be received from the ML model.

The graphical representation of the predicted output formulation properties can be displayed utilizing the user interface. The predicted output formulation properties and the predicted raw materials can be exported to make the predicted output formulation properties and the predicted raw materials (e.g., formulations) and their corresponding weight values available outside the user interface. The predicted output formulation properties and the predicted raw materials can be stored by the ML model as a historical formulation.

In various examples, an output formulation selection, a number of output formulation property selections, and/or a number of raw material selections can be received. The output formulation selection, the number of output formulation property selections, and/or the number of raw material selections can be received via a user interface.

The output formulation selection, the number of output formulation property selections, and/or the number of raw material selections can be provided to a ML model. The ML model can comprise a forward model and an inverse model that predict output formulation properties and raw materials. The predicted output formulation properties and the predicted raw materials for the output formulation can be received from the ML model. The predicted output formulation properties and the predicted raw materials can be provided to the user interface for display.

The output formulation can be a polyurethane foam. The output formulation selection can be the polyurethan foam used for comfort or insulation. A historical formulation selection can be received from the user interface. The predicted output formulation properties and the predicted raw materials can be received from the ML model. The ML model can search historical data set using the forward model and the inverse model.

A predicted formulation selection can be received from the user interface. For example, a predicted formulation can be selected from the predicted formulation provided by the ML model. The predicted output formulation properties and the predicted raw materials can be received from the ML model wherein the ML model predicts the predicted output formulation properties and the predicted raw material using machine learning operations implemented by the forward model and the inverse model.

A units selection can also be received. The units selection can be provided to the ML model. The output formulation properties and the output formulations can be received in the units selected. A range for one or more of the number of output formulation property selections can be received from the ML model. The number of output formulation property selections can comprise one or more of a gel time property, a density free rise property, a density core property, a compression strength property, a lambda property, and/or a min fill property. A minimum quantity of the output formulation selection and a maximum quantity of the output formulation selection can be received from the user interface.

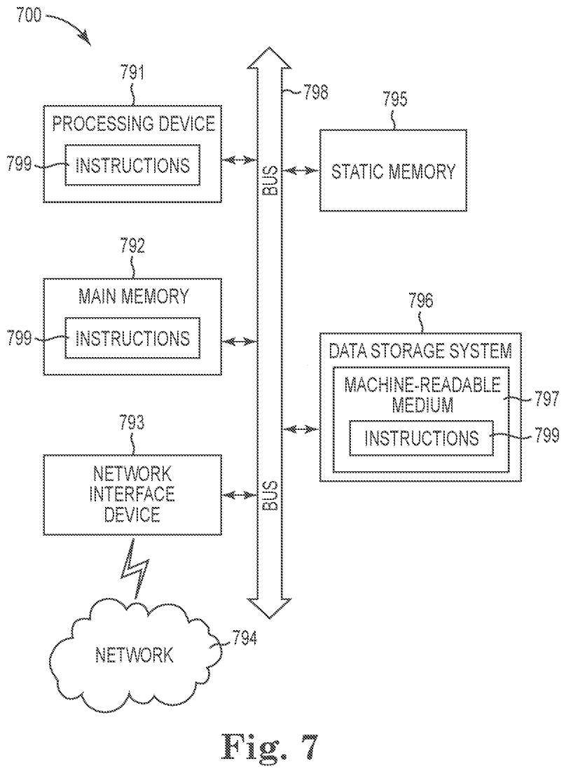

FIG. 7 illustrates an example machine 700 within which a set of instructions, for causing the machine 700 to perform various methodologies discussed herein, can be executed.

The machine 700 can be connected (e.g., networked) to other machines in a LAN, an intranet, an extranet, and/or the Internet. The machine 700 can operate in the capacity of a server or a client machine in client-server network environment, as a peer machine in a peer-to-peer (or distributed) network environment, or as a server or a client machine in a cloud computing infrastructure or environment.

The machine 700 can be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine. Further, while a single machine 700 is illustrated, the term “machine” shall also be taken to include any collection of machines that individually or jointly execute a set (or multiple sets) of instructions to perform any one or more of the methodologies discussed herein.

The example machine 700 includes a processing device 791, a main memory 792 (e.g., read-only memory (ROM), flash memory, dynamic random access memory (DRAM) such as synchronous DRAM (SDRAM) or Rambus DRAM (RDRAM), etc.), a static memory 795 (e.g., flash memory, static random access memory (SRAM), etc.), and a data storage system 796, which communicate with each other via a bus 798.

The processing device 791 represents one or more general-purpose processing devices such as a microprocessor, a central processing unit (CPU), or the like. More particularly, the processing device can be a complex instruction set computing (CISC) microprocessor, reduced instruction set computing (RISC) microprocessor, very long instruction word (VLIW) microprocessor, or a processor implementing other instruction sets, or processors implementing a combination of instruction sets. The processing device 791 can also be one or more special-purpose processing devices such as an application specific integrated circuit (ASIC), a field programmable gate array (FPGA), a digital signal processor (DSP), network processor, or the like. The processing device 791 is configured to execute instructions 799 for performing the operations and steps discussed herein. The machine 700 can further include a network interface device 793 to communicate over the network 794.

The data storage system 796 can include a machine-readable storage medium 797 (also known as a computer-readable medium) on which is stored one or more sets of instructions 799 or software embodying any one or more of the methodologies or functions described herein. The instructions 799 can also reside, completely or at least partially, within the main memory 792 and/or within the processing device 791 during execution thereof by the machine 700, the main memory 792 and the processing device 791 also constituting machine-readable storage media.

In one embodiment, the instructions 799 include instructions to implement functionality corresponding to an interface for generating predictions of raw materials and output formulation properties. While the machine-readable storage medium 797 is shown in an example embodiment to be a single medium, the term “machine-readable storage medium” should be taken to include a single medium or multiple media that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include, but not be limited to, solid-state memories, optical media, and magnetic media.

Although specific embodiments have been described above, these embodiments are not intended to limit the scope of the present disclosure, even where only a single embodiment is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed herein (either explicitly or implicitly), or any generalization thereof, whether or not it mitigates any or all of the problems addressed herein. Various advantages of the present disclosure have been described herein, but embodiments may provide some, all, or none of such advantages, or may provide other advantages.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. An apparatus comprising:
a processing resource configured to:
receive an output formulation selection;
receive a number of output formulation property selections;
receive a number of raw material selections, wherein the output formulation selection, the number of output formulation property selections, and the number of raw material selections are received via a user interface;
provide the output formulation selection, the number of output formulation property selections, and the number of raw material selections to a machine learning (ML) model, wherein the ML model comprises a forward model and an inverse model that predict output formulation properties and raw materials;
receive the predicted output formulation properties and the predicted raw materials for the output formulation from the ML model; and
provide the predicted output formulation properties and the predicted raw materials to the user interface for display.

2. The apparatus of claim 1, wherein the output formulation is a polyurethane foam or a non-cellular material and the output formulation selection is the polyurethane foam used for comfort or insulation or other applications.

3. The apparatus of claim 1, wherein the processing resource is further configured to:
receive a historical formulation selection from the user interface; and
receive the predicted output formulation properties and the predicted raw materials from the ML model wherein the ML model searches historical data set using the forward model and the inverse model.

4. The apparatus of claim 1, wherein the processing resource is further configured to:
receive a predicted formulation selection from the user interface; and
receive the predicted output formulation properties and the predicted raw materials from the ML model.

5. The apparatus of claim 1, wherein the processing resource is further configured to:
receive a units selection;
provide the units selection to the ML model; and
receive the output formulation properties and the output formulations in the units selection.

6. The apparatus of claim 1, wherein the processing resource is further configured to receive a range for one or more of the number of output formulation property selections.

7. The apparatus of claim 6, wherein the number of output formulation property selections comprise one or more of a gel time property, a density free rise property, a density core property, a compression strength property, a lambda property, and a min fill property.

8. The apparatus of claim 1, wherein the processing resource is further configured to receive a minimum quantity of the output formulation selection and a maximum quantity of the output formulation selection.

9. A method, comprising:
receiving, at a processing resource, an output formulation selection;
receiving, at the processing resource, a number of output formulation property selections;
receiving, at the processing resource, a number of raw materials selections, wherein the output formulation selection, the number of output formulation property selections, and the number of raw material selections are received via a user interface;
providing, utilizing a network interface coupled to the processing resource, the output formulation selection, the number of output formulation property selections, and the number of raw material selections to a machine learning (ML) model via the network interface,
wherein the ML model comprises a forward model and an inverse model that predict output formulation properties and raw materials, and
wherein the ML model is implemented in a cloud environment;
running the ML model via the user interface;
receiving the predicted output formulation properties and the predicted raw materials for an output formulation from the ML model; and
providing the predicted output formulation properties and the predicted raw materials to the user interface for display.

10. The method of claim 9, further comprising:
receiving feedback material properties responsive to generating the output formulation using the predicted raw materials; and
provide the feedback material properties to the ML model for training.

11. The method of claim 9, further comprising, responsive to receipt of the raw materials, receiving updated raw materials and corresponding weights percentages of the updated raw materials.

12. The method of claim 11, further comprising:
providing, via the network interface, the updated raw materials to the ML model; and
receiving updated predicted output formulation properties and updated predicted raw materials from the ML model.

13. The method of claim 9, further comprising displaying, via the user interface, a graphical representation of the predicted output formulation properties.

14. The method of claim 9, further comprising exporting the predicted output formulation properties and the predicted raw materials.

15. The method of claim 9, wherein the predicted output formulation properties and the predicted raw materials are stored by the machine learning model as a historical formulation.
